(19) United States
(12) Patent Application Publication
(10) Pub. No.: US 2012/0273771 A1
(43) Pub. Date: Nov. 1, 2012

(54) COMPOUND FOR ORGANIC PHOTOELECTRIC DEVICE AND ORGANIC PHOTOELECTRIC DEVICE INCLUDING THE SAME

(76) Inventors: Ho-Kuk JUNG, Uiwang-si (KR); Dong-Min KANG, Uiwang-si (KR); Kyu Yeol IN, Uiwang-si (KR); Nam-Soo KIM, Uiwang-si (KR); Sung-Hyun JUNG, Uiwang-si (KR); Myeong-Soon KANG, Uiwang-si (KR); Nam-Heon LEE, Uiwang-si (KR); Eui-Su KANG, Uiwang-si (KR); Mi-Young CHAE, Uiwang-si (KR)

(21) Appl. No.: 13/537,952

(22) Filed: Jun. 29, 2012

Related U.S. Application Data
(63) Continuation of application No. PCT/KR2010/007763, filed on Nov. 4, 2010.

(30) Foreign Application Priority Data
Dec. 31, 2009 (KR) 10-2009-0136182

(51) Int. Cl. H01L 51/54; C07D 471/04; H01L 51/46; C07D 401/14

(52) U.S. Cl. 257/40; 544/333; 546/121; 546/144; 257/E51.018; 257/E51.012

(57) ABSTRACT

A compound for an organic photoelectric device is represented by Chemical Formula 1,

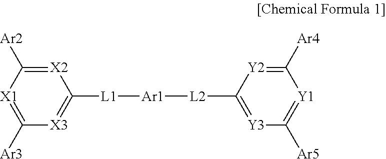

wherein in Chemical Formula 1, Ar1 is a substituted or unsubstituted C2 to C30 heteroarylene group, Ar2 to Ar5 are independently a substituted or unsubstituted C6 to C30 aryl group, L1 and L2 are independently a substituted or unsubstituted C6 to C30 arylene group, X1 to X3 are independently a heteroatom or C—H, provided that at least one of X1 to X3 is a heteroatom, and Y1 to Y3 are independently a heteroatom or C—H, provided that at least one of Y1 to Y3 is a heteroatom.

### CROSS-REFERENCE TO RELATED APPLICATION

This application is a continuation of pending International Application No. PCT/KR2010/007763, entitled “COMPOUND FOR ORGANIC PHOTOELECTRIC DEVICE AND ORGANIC PHOTOELECTRIC DEVICE INCLUDING THE SAME”, which was filed on Nov. 4, 2010, the entire contents of which are hereby incorporated by reference.

### BACKGROUND

1. Field of the Invention

Embodiments relate to a compound for an organic photoelectric device and an organic photoelectric device including the same.

2. Description of the Related Art

An organic photoelectric device may be a device for transforming photo-energy to electrical energy, or conversely, a device for transforming electrical energy to photo-energy.

Organic photoelectric devices may be classified variously in accordance with their driving principle. An organic photoelectric device according to one type may be an electron device driven as follows: excitons may be generated in an organic material layer by photons entering the device from an external light source; the excitons may be separated into electrons and holes, and the electrons and holes may be respectively transferred to different electrodes and used as a current source (a voltage source).

A second organic photoelectric device according to another type may be an electron device driven as follows: a voltage or a current may be applied to at least two electrodes to inject holes and/or electrons into an organic material semiconductor positioned on an interface of the electrodes, and the injected electrons and holes may drive the device.

### SUMMARY

Embodiments are directed to a compound for an organic photoelectric device, the compound being represented by the following Chemical Formula 1.

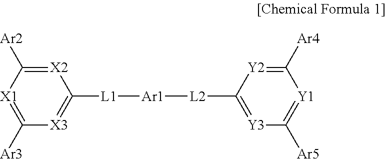

In Chemical Formula 1, Ar1 is a substituted or unsubstituted C2 to C30 heteroarylene group, Ar2 to Ar5 are independently a substituted or unsubstituted C6 to C30 aryl group, L1 and L2 are independently a substituted or unsubstituted C6 to C30 arylene group, X1 to X3 are independently a heteroatom or C—H, provided that at least one of X1 to X3 is a heteroatom, Y1 to Y3 are independently a heteroatom or C—H, provided that at least one of Y1 to Y3 is a heteroatom.

Ar1 may be selected from the group of a substituted or unsubstituted imidazolylene group, a substituted or unsubstituted thiazolylene group, a substituted or unsubstituted oxazolylene group, a substituted or unsubstituted oxadiazolylene group, a substituted or unsubstituted triazolylene group, a substituted or unsubstituted pyrimidinylene group, a substituted or unsubstituted pyridinylene group, a substituted or unsubstituted pyradazinylene group, a substituted or unsubstituted quinolinylene group, a substituted or unsubstituted isoquinolinylene group, a substituted or unsubstituted acridinylene group, a substituted or unsubstituted imidazopyridinylene group, and a substituted or unsubstituted imidazopyrimidinylene group.

Ar1 may be a substituent selected from the group of the following Chemical Formulae 2 to 7.

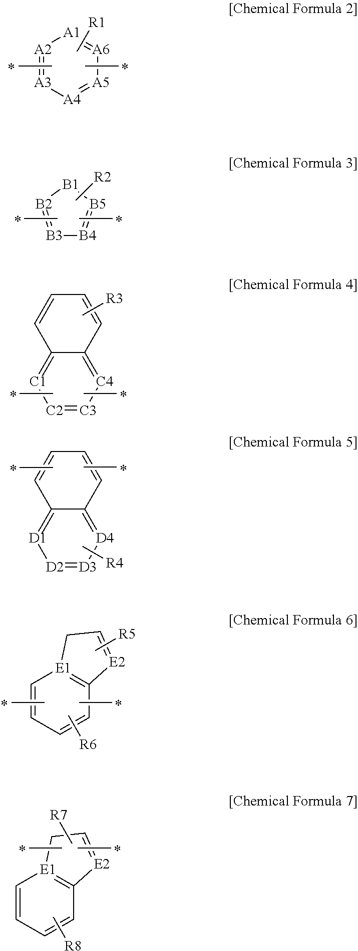

In Chemical Formulae 2 to 7, A1 to A6 may be independently a heteroatom or C—H, provided that at least one of A1 to A6 is a heteroatom, B1 to B5 may be independently a heteroatom or C—H, provided that at least one of B1 to B5 is a heteroatom, C1 to C4 may be independently a heteroatom or C—H, provided that at least one of C1 to C4 is a heteroatom, D1 to D4 may be independently a heteroatom or C—H, provided that at least one of D1 to D4 is a heteroatom, E1 and E2 may be independently a heteroatom or C—H, provided that at least one of E1 and E2 is a heteroatom, and R1 to R8 may be independently selected from the group of hydrogen, a substituted or unsubstituted C1 to C10 alkyl group, a substituted or unsubstituted C5 to C30 aryl group, and a substituted or unsubstituted C2 to C30 heteroaryl group.

Ar2 to Ar5 may be independently selected from the group of a substituted or unsubstituted phenyl group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted perylenyl group, and a substituted or unsubstituted chrysenyl group.

L1 and L2 may be independently selected from the group of a substituted or unsubstituted phenylene group, a substituted or unsubstituted naphthylene group, a substituted or unsubstituted anthracenylene group, a substituted or unsubstituted phenanthrenylene group, a substituted or unsubstituted pyrenylene group, a substituted or unsubstituted perylenylene group, and a substituted or unsubstituted chrysenylene group.

Embodiments are also directed to an organic photoelectric device that includes an anode, a cathode, and an organic thin layer between the anode and the cathode, wherein the organic thin layer includes the compound for an organic photoelectric device.

The organic thin layer may be selected from the group of an emission layer, a hole transport layer (HTL), a hole injection layer (HIL), an electron transport layer (ETL), an electron injection layer (EIL), a hole blocking layer, and a combination thereof.

The compound for an organic photoelectric device may be included in an electron transport layer (ETL) or an electron injection layer (EIL).

The compound for an organic photoelectric device may be included in an emission layer.

The compound for an organic photoelectric device may be used as a phosphorescent or fluorescent host material in an emission layer.

The compound for an organic photoelectric device may be used as a fluorescent blue dopant material in an emission layer.

The organic photoelectric device may be selected from the group of an organic light emitting diode, an organic solar cell, an organic transistor, an organic photo conductor drum, and an organic memory device.

Embodiments are also directed to a display device including the organic photoelectric device.

### BRIEF DESCRIPTION OF THE DRAWINGS

Features will become apparent to those of skill in the art by describing in detail exemplary embodiments with reference to the attached drawings in which:

FIGS. 1 to 5 illustrate cross-sectional views showing organic photoelectric devices including the compound for an organic photoelectric device according to various embodiments.

FIG. 6 illustrates current density changes depending on voltage change according to the Examples and the Comparative Example.

FIG. 7 illustrates luminance changes depending on voltage change according to the Examples and the Comparative Example.

FIG. 8 illustrates luminous efficiency experimental data according to the Examples and the Comparative Example.

FIG. 9 illustrates shows electric power efficiency experimental data according to the Examples and the Comparative Example.

FIG. 10 illustrates life-span measurements and experimental data of the organic photoelectric devices according to the Examples and the Comparative Example.

### DETAILED DESCRIPTION

Korean Patent Application No. 10-2009-0136182, filed on Dec. 31, 2009, in the Korean Intellectual Property Office, and entitled: “Compound for Organic Photoelectric Device and Organic Photoelectric Device Including the Same,” is incorporated by reference herein in its entirety.

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the invention to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

Throughout the specification, the term “substituted” may refer to one substituted with a C1 to C30 alkyl group, a C1 to C10 alkylsilyl group, a C3 to C30 cycloalkyl group, a C6 to C30 aryl group, a C1 to C10 alkoxy group, a fluoro group, a C1 to C10 trifluoroalkyl group such as a trifluoromethyl group, and the like, or a cyano group.

Throughout the specification, the term “hetero” may refer to one including 1 to 3 heteroatoms selected from the group of N, O, S, and P and carbons in the rest thereof, in one ring.

Throughout the specification, the term “a combination thereof” refers to at least two substituents bound to each other by a linker or at least two substituents fused to each other.

Throughout the specification, when a definition is not otherwise provided, the term “alkyl” refers to an aliphatic hydrocarbon group. The alkyl may be a saturated alkyl group that does not include any alkene or alkyne. The alkyl may be branched, linear, or cyclic.

The term “alkene” refers to a group in which at least two carbon atoms are bound in at least one carbon-carbon double bond, and the term “alkyne” refers to a group in which at least two carbon atoms are bound in at least one carbon-carbon triple bond.

The alkyl group may have 1 to 20 carbon atoms. The alkyl group may be a medium-sized alkyl having 1 to 10 carbon atoms. The alkyl group may be a lower alkyl having 1 to 6 carbon atoms.

For example, a C1 to C4 alkyl may have 1 to 4 carbon atoms and may be selected from the group of methyl, ethyl, propyl, iso-propyl, n-butyl, iso-butyl, sec-butyl, and t-butyl.

Examples of an alkyl group may be selected from the group of methyl, ethyl, propyl, isopropyl, butyl, isobutyl, t-butyl, pentyl, hexyl, ethenyl, propenyl, butenyl, cyclopropyl, cyclobutyl, cyclopentyl, cyclohexyl, or the like, which may be individually and independently substituted.

The term “aryl” may refer to an aryl group including a carbocyclic aryl (e.g., phenyl) having at least one ring having a covalent pi electron system. The term also may refer to monocyclic or fused polycyclic (i.e., rings sharing adjacent pairs of carbon atoms) groups. In addition, this term may also refer to a spiro compound having a contact point of one carbon.

The term “heteroaryl” may refer to a heterocyclic aryl group including a carbocyclic aryl (e.g., pyridine) having at least one ring having a covalent pi electron system. The term also may refer to monocyclic or fusion ring polycyclic (i.e., groups sharing adjacent pairs of carbon atoms) groups. In addition, the term may also refer to a spiro compound having a contact point of one carbon.

According to an embodiment, a compound for an organic photoelectric device may have a structure that a substituent represented by at least two “*-arylene group-heteroaryl groups” is combined with a core of a heteroarylene group. In the specification, “*” marks where a substituent is combined.

In addition, the compound for an organic photoelectric device may have a core structure and include various substituents for the two substituents and, thus, may include various energy band gaps. Therefore, the compound may satisfy conditions required for an emission layer, as well as an electron injection layer (EIL) and an electron transfer layer.

When a compound having appropriate energy levels due to substituents is used for an organic photoelectric device, electron transfer capability may be reinforced and, thus, excellent efficiency and driving voltage and excellent electrochemical and thermal stability may be improved, resulting in improvement of life-span characteristics of the organic photoelectric device.

According to an embodiment, a compound for an organic photoelectric device represented by the following Chemical Formula 1 is provided.

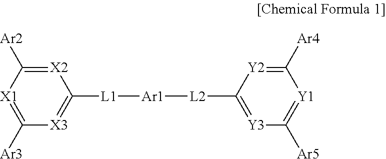

In Chemical Formula 1, Ar1 may be a substituted or unsubstituted C2 to C30 heteroarylene group. For example, Ar1 may be selected from the group of a substituted or unsubstituted imidazolylene group, a substituted or unsubstituted thiazolylene group, a substituted or unsubstituted oxazolylene group, a substituted or unsubstituted oxadiazolylene group, a substituted or unsubstituted triazolylene group, a substituted or unsubstituted pyrimidinylene group, a substituted or unsubstituted pyridinylene group, a substituted or unsubstituted pyradazinylene group, a substituted or unsubstituted quinolinylene group, a substituted or unsubstituted isoquinolinylene group, a substituted or unsubstituted acridinylene group, a substituted or unsubstituted imidazopyridinylene group, and substituted or unsubstituted imidazopyrimidinylene group.

As specific examples, Ar1 may be a substituent selected from the group of the following Chemical Formulae 2 to 7.

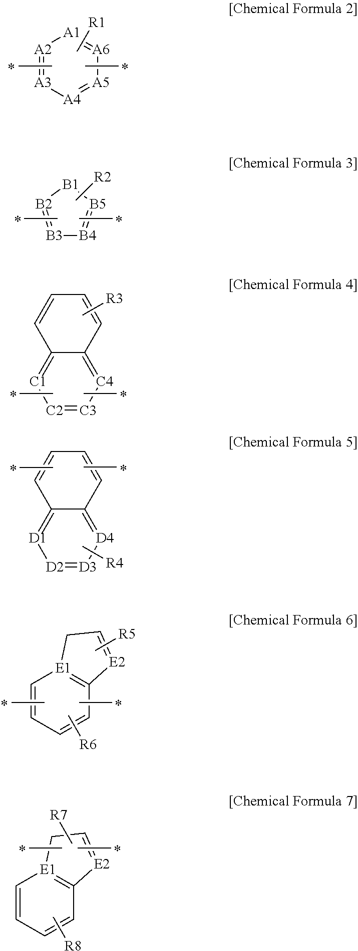

In Chemical Formulae 2 to 7, A1 to A6 may be independently a heteroatom or C—H, provided that at least one of A1 to A6 is a heteroatom, B1 to B5 may be independently a heteroatom or C—H, provided that at least one of B1 to B5 is a heteroatom, C1 to C4 may be independently a heteroatom or C—H, provided that at least one of C1 to C4 is a heteroatom, D1 to D4 may be independently a heteroatom or C—H, provided that at least one of D1 to D4 is a heteroatom, E1 and E2 may be independently a heteroatom or C—H, provided that at least one of E1 and E2 is a heteroatom, and R1 to R8 may be independently selected from the group of hydrogen, a substituted or unsubstituted C1 to C10 alkyl group, a substituted or unsubstituted C5 to C30 aryl group, and a substituted or unsubstituted C2 to C30 heteroaryl group.

The Ar1 substituent is the core, the aforementioned heteroarylene group. As shown in Chemical Formula 1, L1 and L2 are combined with the core.

L1 and L2 may be independently a substituted or unsubstituted C6 to C30 arylene group. For example, L1 and L2 may be independently selected from the group of a substituted or unsubstituted phenylene group, a substituted or unsubstituted naphthylene group, a substituted or unsubstituted anthracenylene group, a substituted or unsubstituted phenanthrenylene group, a substituted or unsubstituted pyrenylene group, a substituted or unsubstituted perylenyl group, and a substituted or unsubstituted chrysenylene group.

The L1 and L2 may be adjusted with respect to a π-conjugation length to control light emission in a visible region. Accordingly, the compound may be usefully applied to an emission layer for an organic photoelectric device. When the number of carbons is in the aforementioned range, the compound may have sufficient effects for an organic photoelectric device.

When the compound is used to form an emission layer, a compound with the number of carbons of more than 6 may have appropriate conjugation while a compound with the number of carbons less than 30 may avoid an inappropriate shifting of light-emitting colors toward an infrared region.

In addition, when the compound is used to form an electron transport layer (ETL), a compound having the number of carbons of more than 6 may have appropriate conjugation, while a compound having the number of carbons of less than 30 may avoid a decrease of a band gap and a deterioration of a capability of inhibiting a hole.

As shown in Chemical Formula 1, a heteroaryl group may be independently combined with the L1 and L2.

In Chemical Formula 1, X1 to X3 may be independently a heteroatom or C—H, provided that at least one of X1 to X3 is a heteroatom, and Y1 to Y3 may be independently a heteroatom or C—H, provided that at least one of Y1 to Y3 is a heteroatom.

When the compound has a structure of combining the heteroaryl group, the compound may have excellent thermal stability due to hydrogen bonds of hetero atoms, improving life-span characteristics of an organic photoelectric device.

In addition, Ar2 to Ar5, which are substituents combined with heteroaryl groups at the end, may be independently a substituted or unsubstituted C6 to C30 aryl group. For example, Ar2 to Ar5 may be independently selected from the group of a substituted or unsubstituted phenyl group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted perylenyl group, and a substituted or unsubstituted chrysenyl group.

Various electron transfer capabilities of a compound may be adjusted according to a selection of Ar2 to Ar5. In addition, crystallinity of the compound may be adjusted, thereby providing a long life-span for a device.

The compound for an organic photoelectric device may be represented by the following Chemical Formulae 8 to 18, as non-limiting examples of the compound of Formula 1. The combination of the substituents may be represented by the following compounds 1 to 385.

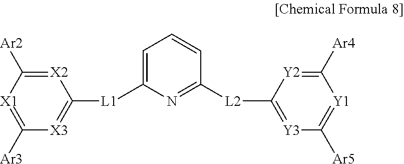

In Chemical Formula 8, Ar2 to Ar5, X1 to X3, Y1 to Y3, L1, and L2 are the same as defined in the following Table 1.

TABLE 1

| Compound | Ar2 and Ar4 | Ar3 and Ar5 | L1 and L2 | X1 and Y1 | X2 and Y2 | X3 and Y3 |
|---|---|---|---|---|---|---|
| 1 2 3 4 5 6 7 | 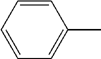 | 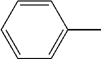 | 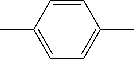 | N C C N N C N | C N C N C N N | C C N C N N N |
| 8 9 10 11 12 13 14 | 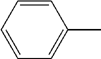 | 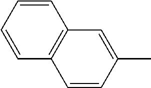 | 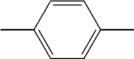 | N C C N N C N | C N C N C N N | C C N C N N N |
| 15 16 17 18 19 20 21 | 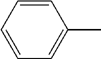 | 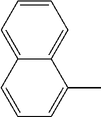 | 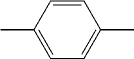 | N C C N N C N | C N C N C N N | C C N C N N N |
| 22 23 24 25 26 27 28 | 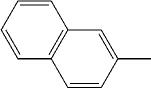 | 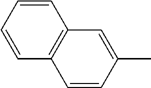 | 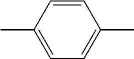 | N C C N N C N | C N C N C N N | C C N C N N N |
| 29 30 31 32 33 34 35 | 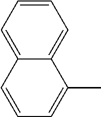 | 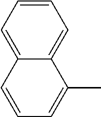 | 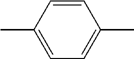 | N C C N N C N | C N C N C N N | C C N C N N N |

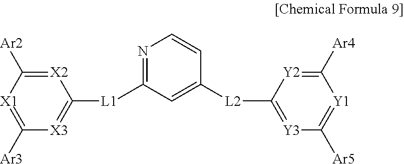

In Chemical Formula 9, Ar2 to Ar5, X1 to X3, Y1 to Y3, L1, and L2 are the same as defined in the following Table 2.

TABLE 2

| Compound | Ar2 and Ar4 | Ar3 and Ar5 | L1 and L2 | X1 and Y1 | X2 and Y2 | X3 and Y3 |
|---|---|---|---|---|---|---|
| 36 37 38 39 40 41 42 | 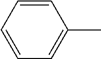 | 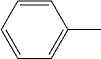 | 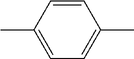 | N C C N N C N | C N C N C N N | C C N C N N N |
| 43 44 45 46 47 48 49 | 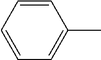 | 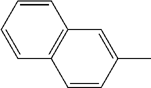 | 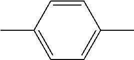 | N C C N N C N | C N C N C N N | C C N C N N N |
| 50 51 52 53 54 55 56 | 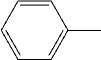 | 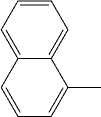 | 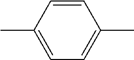 | N C C N N C N | C N C N C N N | C C N C N N N |
| 57 58 59 60 61 62 63 | 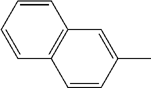 | 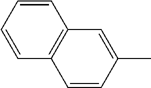 | 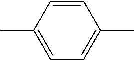 | N C C N N C N | C N C N C N N | C C N C N N N |
| 64 65 66 67 68 69 70 | 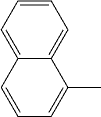 | 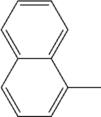 | 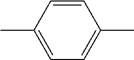 | N C C N N C N | C N C N C N N | C C N C N N N |

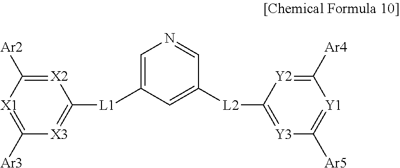

In Chemical Formula 10, Ar2 to Ar5, X1 to X3, Y1 to Y3, L1, and L2 are the same as defined in the following Table 3.

TABLE 3

| Compound | Ar2 and Ar4 | Ar3 and Ar5 | L1 and L2 | X1 and Y1 | X2 and Y2 | X3 and Y3 |
|---|---|---|---|---|---|---|
| 71 72 73 74 75 76 77 | 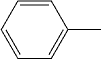 | 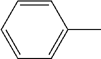 | 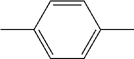 | N C C N N C N | C N C N C N N | C C N C N N N |
| 78 79 80 81 82 83 84 | 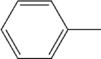 | 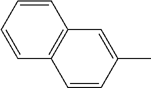 | 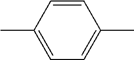 | N C C N N C N | C N C N C N N | C C N C N N N |
| 85 86 87 88 89 90 91 | 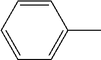 | 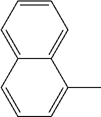 | 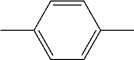 | N C C N N C N | C N C N C N N | C C N C N N N |
| 92 93 94 95 96 97 98 | 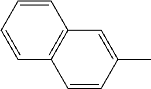 | 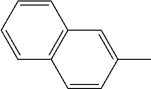 | 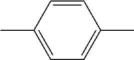 | N C C N N C N | C N C N C N N | C C N C N N N |
| 99 100 101 102 103 104 105 | 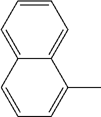 | 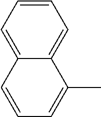 | 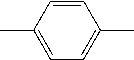 | N C C N N C N | C N C N C N N | C C N C N N N |

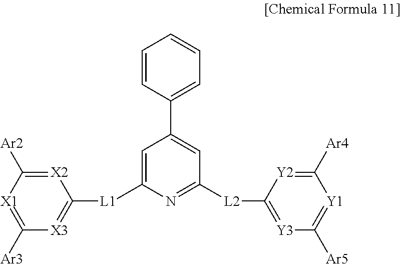

In Chemical Formula 11, Ar2 to Ar5, X1 to X3, Y1 to Y3, L1, and L2 are the same as defined in the following Table 4.

TABLE 4

| compound | Ar2 and Ar4 | Ar3 and Ar5 | L1 and L2 | X1 and Y1 | X2 and Y2 | X3 and Y3 |
|---|---|---|---|---|---|---|
| 106 107 108 109 110 111 112 | 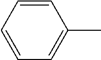 | 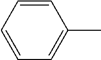 | 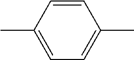 | N C C N N C N | C N C N C N N | C C N C N N N |
| 113 114 115 116 117 118 119 | 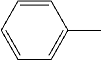 | 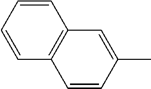 | 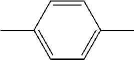 | N C C N N C N | C N C N C N N | C C N C N N N |
| 120 121 122 123 124 125 126 | 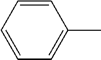 | 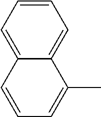 | 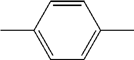 | N C C N N C N | C N C N C N N | C C N C N N N |
| 127 128 129 130 131 132 133 | 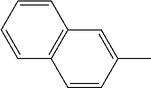 | 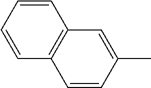 | 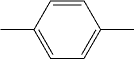 | N C C N N C N | C N C N C N N | C C N C N N N |
| 134 135 136 137 138 139 140 | 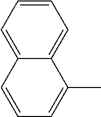 | 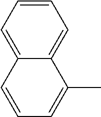 | 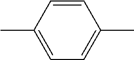 | N C C N N C N | C N C N C N N | C C N C N N N |

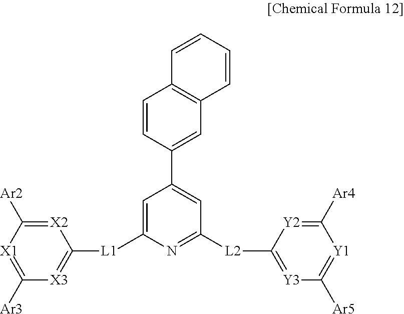

In Chemical Formula 12, Ar2 to Ar5, X1 to X3, Y1 to Y3, L1, and L2 are the same as defined in the following Table 5.

TABLE 5

| Compound | Ar2 and Ar4 | Ar3 and Ar5 | L1 and L2 | X1 and Y1 | X2 and Y2 | X3 and Y3 |
|---|---|---|---|---|---|---|
| 141 142 143 144 145 146 147 | 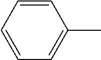 | 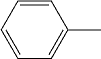 | 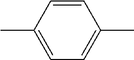 | N C C N N C N | C N C N C N N | C C N C N N N |
| 148 149 150 151 152 153 154 | 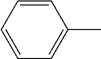 | 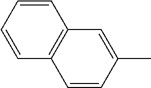 | 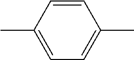 | N C C N N C N | C N C N C N N | C C N C N N N |
| 155 156 157 158 159 160 161 | 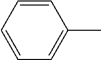 | 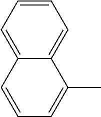 | 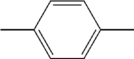 | N C C N N C N | C N C N C N N | C C N C N N N |
| 162 163 164 165 166 167 168 | 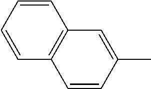 | 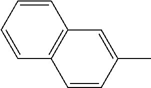 | 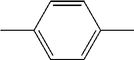 | N C C N N C N | C N C N C N N | C C N C N N N |
| 169 170 171 172 173 174 175 | 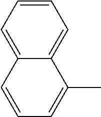 | 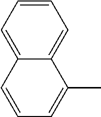 | 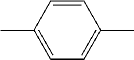 | N C C N N C N | C N C N C N N | C C N C N N N |

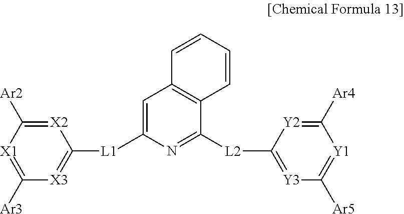

In Chemical Formula 13, Ar2 to Ar5, X1 to X3, Y1 to Y3, L1, and L2 are the same as defined in the following Table 6.

TABLE 6

| Compound | Ar2 and Ar4 | Ar3 and Ar5 | L1 and L2 | X1 and Y1 | X2 and Y2 | X3 and Y3 |
|---|---|---|---|---|---|---|
| 176 177 178 179 180 181 182 | 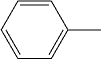 | 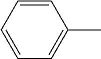 | 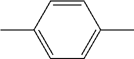 | N C C N N C N | C N C N C N N | C C N C N N N |
| 183 184 185 186 187 188 189 | 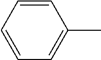 | 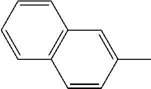 | 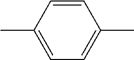 | N C C N N C N | C N C N C N N | C C N C N N N |
| 190 191 192 193 194 195 196 | 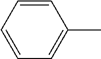 | 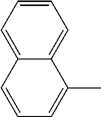 | 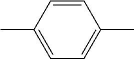 | N C C N N C N | C N C N C N N | C C N C N N N |
| 197 198 199 200 201 202 203 | 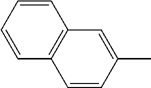 | 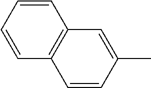 | 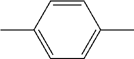 | N C C N N C N | C N C N C N N | C C N C N N N |
| 204 205 206 207 208 209 210 | 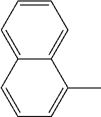 | 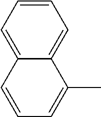 | 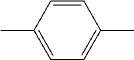 | N C C N N C N | C N C N C N N | C C N C N N N |

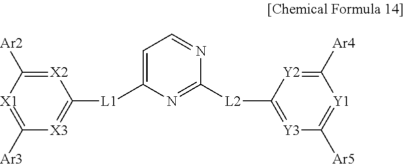

In Chemical Formula 14, Ar2 to Ar5, X1 to X3, Y1 to Y3, L1, and L2 are the same as defined in the following Table 7.

TABLE 7

| Compound | Ar 2 and Ar4 | Ar3 and Ar5 | L1 and L2 | X1 and Y1 | X2 and Y2 | X3 and Y3 |
|---|---|---|---|---|---|---|
| 211 212 213 214 215 216 217 | 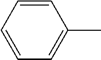 | 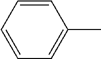 | 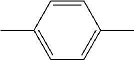 | N C C N N C N | C N C N C N N | C C N C N N N |
| 218 219 220 221 222 223 224 | 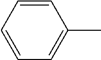 | 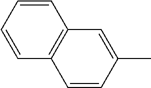 | 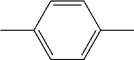 | N C C N N C N | C N C N C N N | C C N C N N N |
| 225 226 227 228 229 230 231 | 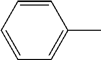 | 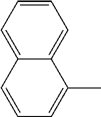 | 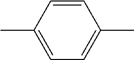 | N C C N N C N | C N C N C N N | C C N C N N N |
| 232 233 234 235 236 237 238 | 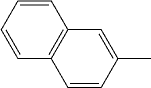 | 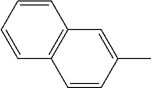 | 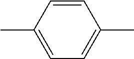 | N C C N N C N | C N C N C N N | C C N C N N N |
| 239 240 241 242 243 244 245 | 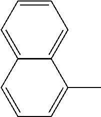 | 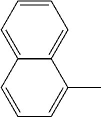 | 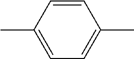 | N C C N N C N | C N C N C N N | C C N C N N N |

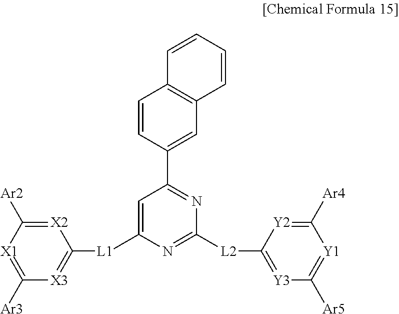

In Chemical Formula 15, Ar2 to Ar5, X1 to X3, Y1 to Y3, L1, and L2 are the same as defined in the following Table 8.

TABLE 8

| Compound | Ar2 and Ar4 | Ar3 and Ar5 | L1 and L2 | X1 and Y1 | X2 and Y2 | X3 and Y3 |
|---|---|---|---|---|---|---|
| 246 247 248 249 250 251 252 | 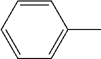 | 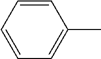 | 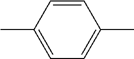 | N C C N N C N | C N C N C N N | C C N C N N N |
| 253 254 255 256 257 258 259 | 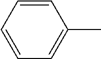 | 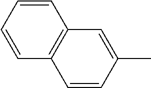 | 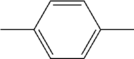 | N C C N N C N | C N C N C N N | C C N C N N N |
| 260 261 262 263 264 265 266 | 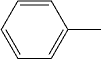 | 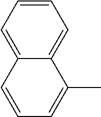 | 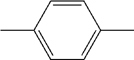 | N C C N N C N | C N C N C N N | C C N C N N N |
| 267 268 269 270 271 272 273 | 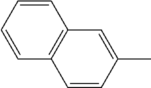 | 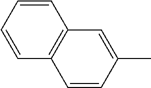 | 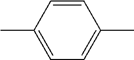 | N C C N N C N | C N C N C N N | C C N C N N N |
| 274 275 276 277 278 279 280 | 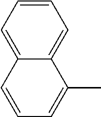 | 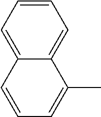 | 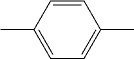 | N C C N N C N | C N C N C N N | C C N C N N N |

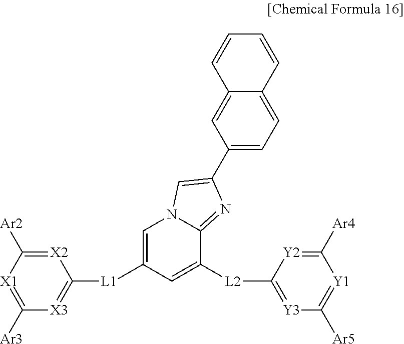

In Chemical Formula 16, Ar2 to Ar5, X1 to X3, Y1 to Y3, L1, and L2 are the same as defined in the following Table 9.

TABLE 9

| Compound | Ar2 and Ar4 | Ar3 and Ar5 | L1 and L2 | X1 and Y1 | X2 and Y2 | X3 and Y3 |
|---|---|---|---|---|---|---|
| 281 282 283 284 285 286 287 | 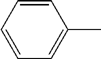 | 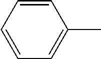 | 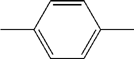 | N C C N N C N | C N C N C N N | C C N C N N N |
| 288 289 290 291 292 293 294 | 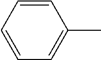 | 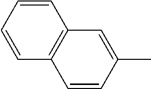 | 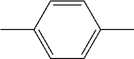 | N C C N N C N | C N C N C N N | C C N C N N N |
| 295 296 297 298 299 300 301 | 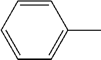 | 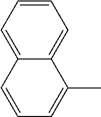 | 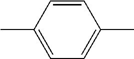 | N C C N N C N | C N C N C N N | C C N C N N N |
| 302 303 304 305 306 307 308 | 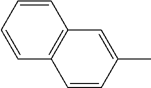 | 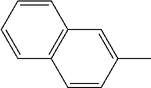 | 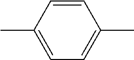 | N C C N N C N | C N C N C N N | C C N C N N N |
| 309 310 311 312 313 314 315 | 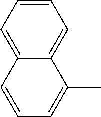 | 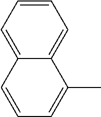 | 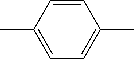 | N C C N N C N | C N C N C N N | C C N C N N N |

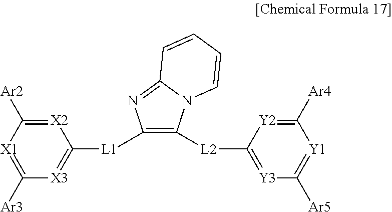

In Chemical Formula 17, Ar2 to Ar5, X1 to X3, Y1 to Y3, L1, and L2 are the same as defined in the following Table 10.

TABLE 10

| compound | Ar2 and Ar4 | Ar3 and Ar5 | L1 and L2 | X1 and Y1 | X2 and Y2 | X3 and Y3 |
|---|---|---|---|---|---|---|
| 316 317 318 319 320 321 322 | 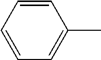 | 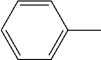 | 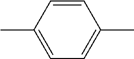 | N C C N N C N | C N C N C N N | C C N C N N N |
| 323 324 325 326 327 328 329 | 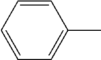 | 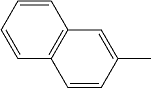 | 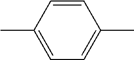 | N C C N N C N | C N C N C N N | C C N C N N N |
| 330 331 332 333 334 335 336 | 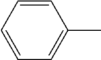 | 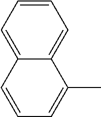 | 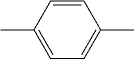 | N C C N N C N | C N C N C N N | C C N C N N N |
| 337 338 339 340 341 342 343 | 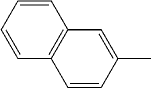 | 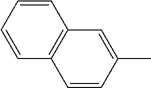 | 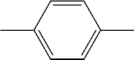 | N C C N N C N | C N C N C N N | C C N C N N N |
| 344 345 346 347 348 349 350 | 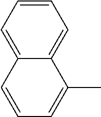 | 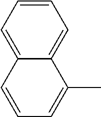 | 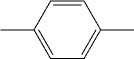 | N C C N N C N | C N C N C N N | C C N C N N N |

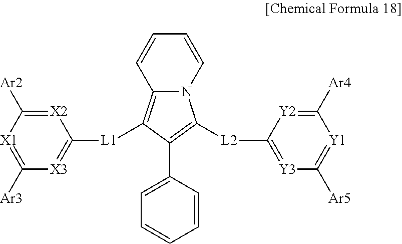

In Chemical Formula 18, Ar2 to Ar5, X1 to X3, Y1 to Y3, L1, and L2 are the same as defined in the following Table 11.

TABLE 11

| Compound | Ar2 and Ar4 | Ar3 and Ar5 | L1 and L2 | X1 and Y1 | X2 and Y2 | X3 and Y3 |
|---|---|---|---|---|---|---|
| 351 352 353 354 355 356 357 | 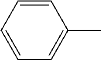 | 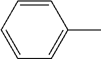 | 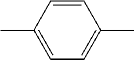 | N C C N N C N | C N C N C N N | C C N C N N N |
| 358 359 360 361 362 363 364 | 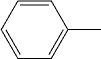 | 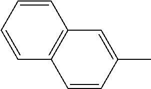 | 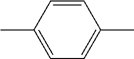 | N C C N N C N | C N C N C N N | C C N C N N N |
| 365 366 367 368 369 370 371 | 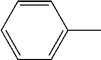 | 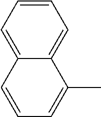 | 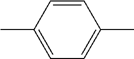 | N C C N N C N | C N C N C N N | C C N C N N N |
| 372 373 374 375 376 377 378 | 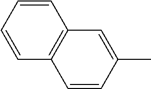 | 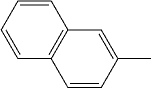 | 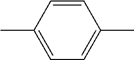 | N C C N N C N | C N C N C N N | C C N C N N N |
| 379 380 381 382 383 384 385 | 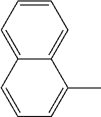 | 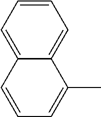 | 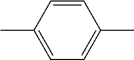 | N C C N N C N | C N C N C N N | C C N C N N N |

The compound for an organic photoelectric device, as described above, may have a glass transition temperature of higher than or equal to 110° C. and a thermal decomposition temperature of higher than or equal to 400° C., so as to improve thermal stability. Thereby, an organic photoelectric device having a high efficiency may be provided.

The compound for an organic photoelectric device, as described above, may play a role of emitting light, or injecting and/or transporting electrons, and may act as a light emitting host together with a suitable dopant. The compound for an organic photoelectric device may be applied as, e.g., a phosphorescent or fluorescent host material, a blue light emitting dopant material, or an electron transport material.

The compound for an organic photoelectric device according to an embodiment may be used for an organic thin layer. Accordingly, the life-span characteristic, efficiency characteristic, electrochemical stability, and thermal stability of an organic photoelectric device may be improved and the driving voltage may be decreased.

According to an embodiment, an organic photoelectric device is provided that includes the compound for an organic photoelectric device. The organic photoelectric device may include an organic photoelectric device, an organic solar cell, an organic transistor, an organic photosensitive drum, an organic memory device, or the like. For example, the compound for an organic photoelectric device according to an embodiment may be included in an electrode or an electrode buffer layer in the organic solar cell to improve the quantum efficiency, and it may be used as an electrode material for a gate electrode, a source-drain electrode, or the like in the organic transistor.

Hereinafter, a detailed description relating to the organic photoelectric device will be provided. According to an embodiment, the organic photoelectric device includes an anode, a cathode, and at least one organic thin layer interposed between the anode and the cathode, wherein the at least one organic thin layer may provide an organic photoelectric device including the compound for an organic photoelectric device according to an embodiment.

The organic thin layer that may include the compound for an organic photoelectric device may include a layer selected from the group of an emission layer, a hole transport layer (HTL), a hole injection layer (HIL), an electron transport layer (ETL), an electron injection layer (EIL), a hole blocking film, and a combination thereof. The at least one layer may include the compound for an organic photoelectric device according to an embodiment. Particularly, the electron transport layer (ETL) or the electron injection layer (EIL) may include the compound for an organic photoelectric device according to an embodiment. In addition, when the compound for an organic photoelectric device is included in the emission layer, the compound for an organic photoelectric device may be included as a phosphorescent or fluorescent host, and particularly, as a fluorescent blue dopant material.

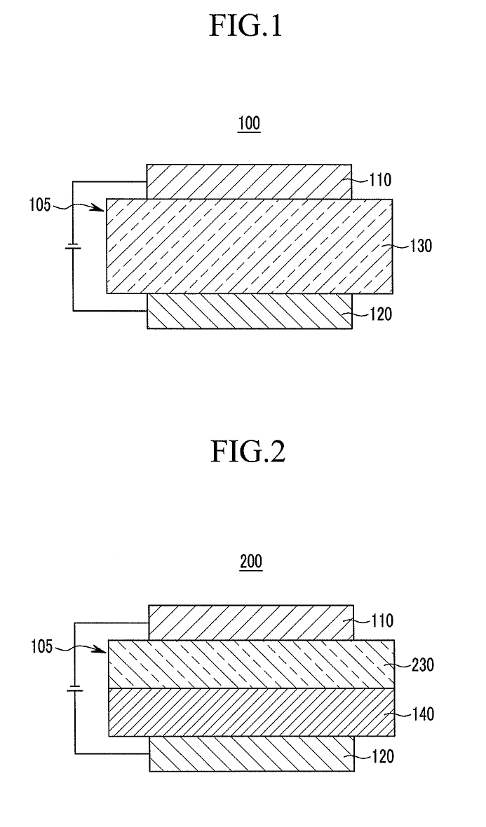

FIGS. 1 to 5 are cross-sectional views showing an organic photoelectric device including the compound for an organic photoelectric device according to an embodiment.

Referring to FIGS. 1 to 5, organic photoelectric devices 100, 200, 300, 400, and 500 according to embodiments include at least one organic thin layer 105 interposed between an anode 120 and a cathode 110.

The anode 120 may include an anode material laving a large work function to assist hole injection into an organic thin layer. The anode material may include: a metal such as nickel, platinum, vanadium, chromium, copper, zinc, or gold, or alloys thereof; a metal oxide such as zinc oxide, indium oxide, indium tin oxide (ITO), or indium zinc oxide (IZO); a combined metal and oxide such as ZnO:Al or SnO₂:Sb; or a conductive polymer such as poly(3-methylthiophene), poly[3,4-(ethylene-1,2-dioxy)thiophene] (PEDT), polypyrrole, or polyaniline, as examples. For example, a transparent electrode may include indium tin oxide (ITO) as an anode.

The cathode 110 may include a cathode material having a small work function to assist electron injection into an organic thin layer. The cathode material may include: a metal such as magnesium, calcium, sodium, potassium, titanium, indium, yttrium, lithium, gadolinium, aluminum, silver, tin, or lead, or alloys thereof; or a multi-layered material such as LiF/Al, Liq/Al, LiO₂/Al, LiF/Ca, LiF/Al, or BaF₂/Ca, as examples. For example, the cathode may be a metal electrode including aluminum.

Referring to FIG. 1, the organic photoelectric device 100 may include an organic thin layer 105 including only an emission layer 130.

Referring to FIG. 2, a double-layered organic photoelectric device 200 may include an organic thin layer 105 including an emission layer 230 including an electron transport layer (ETL), and a hole transport layer (HTL) 140. As shown in FIG. 2, the organic thin layer 105 may include a double layer of the emission layer 230 and hole transport layer (HTL) 140. Referring to FIG. 2, a double-layered organic photoelectric device 200 may include an organic thin layer 105 including an emission layer 230 including an electron transport layer (ETL), and a hole transport layer (HTL) 140.

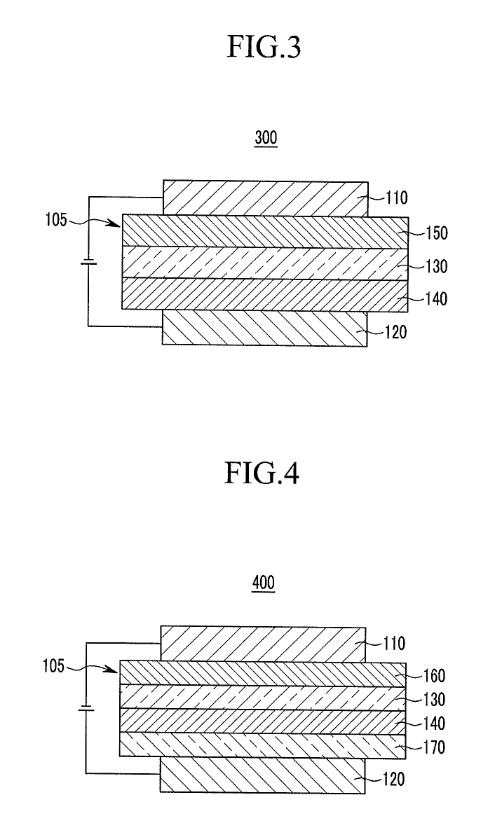

Referring to FIG. 3, a three-layered organic photoelectric device 300 may include an organic thin layer 105 including an electron transport layer (ETL) 150, an emission layer 130, and a hole transport layer (HTL) 140. The emission layer 130 may be independently installed, and layers having an excellent electron transporting property or an excellent hole transporting property may be separately stacked.

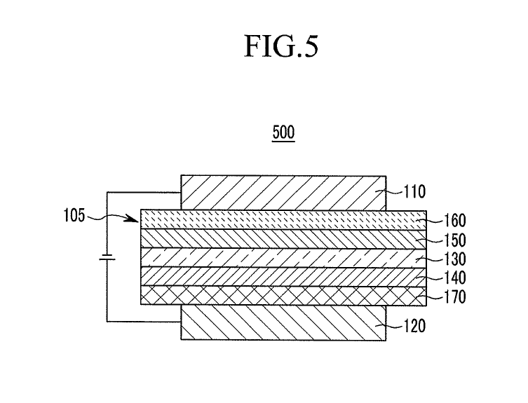

As shown in FIG. 4, a four-layered organic photoelectric device 400 may include an organic thin layer 105 including an electron injection layer (EIL) 160, an emission layer 130, a hole transport layer (HTL) 140, and a hole injection layer (HIL) 170 for binding with the cathode of ITO.

As shown in FIG. 5, a five layered organic photoelectric device 500 may include an organic thin layer 105 including an electron transport layer (ETL) 150, an emission layer 130, a hole transport layer (HTL) 140, and a hole injection layer (HIL) 170, and may further include an electron injection layer (EIL) 160 to achieve a low voltage.

In FIGS. 1 to 5, the organic thin layer 105 including at least one selected from the group of an electron transport layer (ETL) 150, an electron injection layer (EIL) 160, an emission layer 130 and 230, a hole transport layer (HTL) 140, a hole injection layer (HIL) 170, and combinations thereof includes the compound for an organic photoelectric device. The compound for the organic photoelectric device may be used for an electron transport layer (ETL) 150 or an electron injection layer (EIL) 160. When it is used for the electron transport layer (ETL), it may be possible to provide an organic photoelectric device having a more simple structure because an additional hole blocking layer (not shown) may not be required.

Furthermore, when the compound for an organic photoelectric device is included in the emission layers 130 and 230, the material for the organic photoelectric device may be included as a phosphorescent or fluorescent host or a fluorescent blue dopant.

The organic photoelectric device may be fabricated by forming an anode on a substrate; forming an organic thin layer in accordance with a dry coating method such as evaporation, sputtering, plasma plating, and ion plating or a wet coating method such as spin coating, dipping, and flow coating; and providing a cathode thereon.

Another embodiment provides a display device including the organic photoelectric device described above.

Hereinafter, the embodiments are illustrated in more detail with reference to examples. However, the following are exemplary embodiments and are not limiting.

(Preparation of Compound for Organic Photoelectric Device)

### Example 1

### Synthesis of Compound 246

The compound 246, as an example, was synthesized according to Reaction Scheme 1.

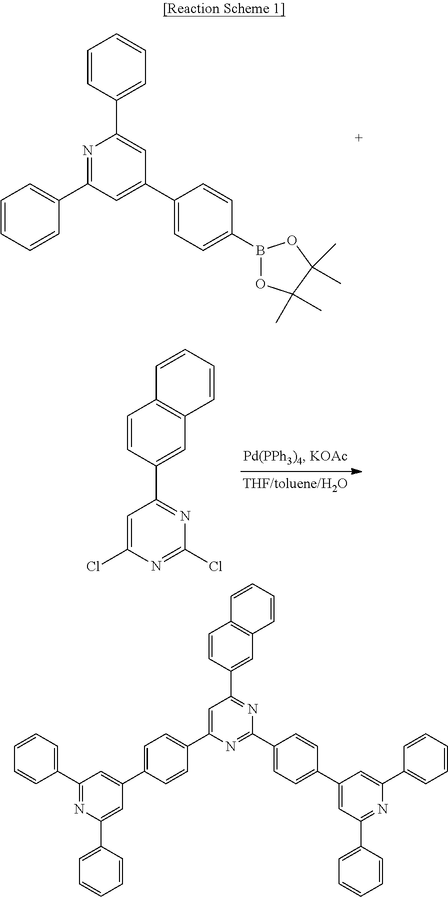

7 g (25 mmol) of 2,4-dichloro-6-(naphthalen-7-yl)pyrimidine, 24.3 g (56 mmol) of 4-(4-(4,4,5,5-tetramethyl-1,3,2-dioxaborolan-2-yl)phenyl)-2,6-diphenylpyridine, and 1.5 g (1.3 mmol) of tetrakis-(triphenylphosphine)palladium were suspended in a mixed solvent of 210 ml of tetrahydrofuran and 140 mL of toluene, and a solution prepared by dissolving 14.1 g (100 mmol) of potassium acetate in 140 mL of water was added thereto. The mixture was heated and refluxed for 12 hours. The liquid reactant was separated into two layers, and the organic layer thereof was cleaned with a sodium chloride saturated aqueous solution and dried with anhydrous sodium sulfate. The organic solvent therein was distilled and removed under a reduced pressure, and the residue was recrystallized with toluene. Then, the precipitate was separated with a filter and cleaned with toluene, obtaining 16.7 g of a compound (yield: 80.3%).

(Calculation value: 816.99/Measurement value: MS[M+1]816)

### Example 2

### Synthesis of Compound 248

The compound 248, as an example, was synthesized according to the following Reaction Scheme 2.

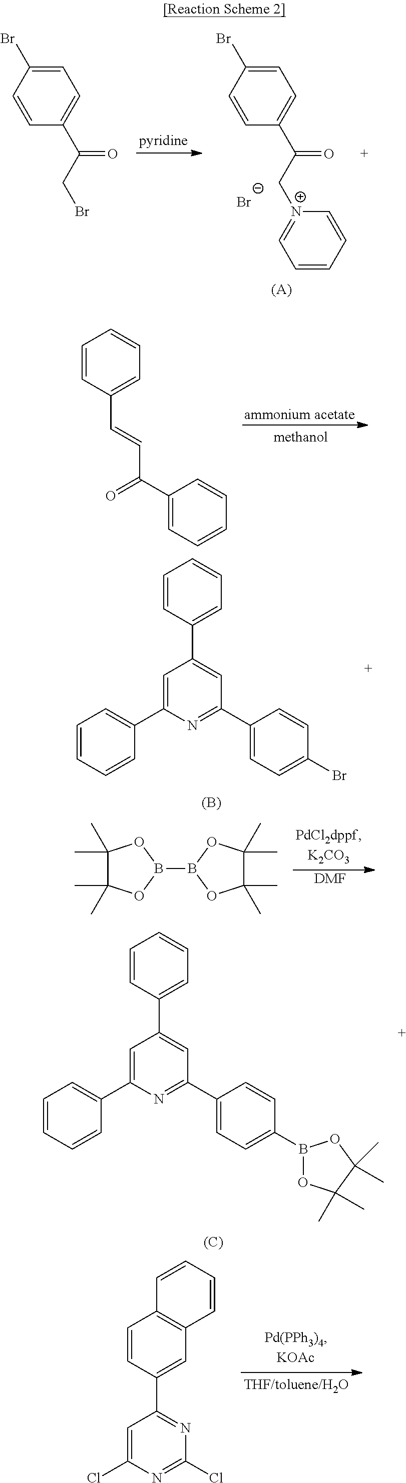

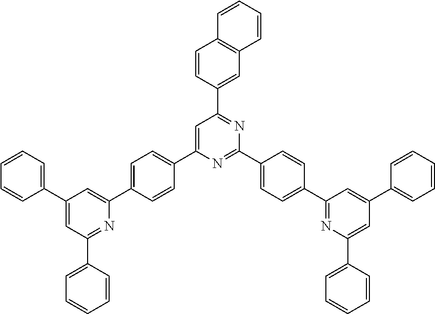

First Step; Synthesis of Intermediate Product (A)

100 g (360 mmol) of 4-bromophenacyl bromide was slowly put into 1000 mL of pyridine, and the mixture was agitated at room temperature for 1 hour. The precipitated solid was filtered and cleaned with diethyl ether, obtaining 127.4 g of an intermediate product A (yield: 99%).

Second Step; Synthesis of Intermediate Product (B)

185.2 g (86.4 mmol) of the intermediate product A, 90 g (43.2 mmol) of trans chalcone, and 333 g (432 mmol) of ammonium acetate were suspended in 1200 mL of methanol, and the mixture was heated and refluxed for 12 hours. After cooling down the reactant, the precipitated solid was filtered and cleaned with methanol, obtaining 88.4 g of an intermediate product (B) (yield: 53%).

Third Step: Synthesis of Intermediate Product (C)

130 g (337 mmol) of the intermediate product (B), 102.7 g (404.4 mmol) of bis(pinacolato)diboron, 6.9 g (8.4 mmol) of [1,1′-bis(diphenylphosphino)ferrocene]dichloropalladium (II), and 99.2 g (1011 mmol) of potassium carbonate were suspended in 650 mL of dimethylformamide. The suspended reactant was agitated at 80° C. for 12 hours. After cooling the reactant, the reactant was poured into distilled water to precipitate a solid. The solid was filtered and separated. The filtered solid was recrystallized with ethylacetate/hexane, obtaining 88.4 g of an intermediate product (C) (yield: 53%).

Fourth Step: Synthesis of Compound 248

2.5 g (9.1 mmol) of 2,4-dichloro-6-(naphthalen-7-yl)pyrimidine, 8.7 g (20 mmol) of the intermediate product (C), and 0.5 g (0.5 mmol) of tetrakis-(triphenylphosphine)palladium were suspended in a mixed solvent of 75 mL of tetrahydrofuran and 50 mL of toluene, and a solution prepared by dissolving 5 g (36.4 mmol) of potassium acetate in 140 mL of water was added thereto. The mixture was heated and refluxed for 12 hours. The liquid reactant was separated into two layers, and the organic layer thereof was cleaned with a sodium chloride saturated aqueous solution and dried with anhydrous sodium sulfate. Then, an organic solvent therein was removed under a reduced pressure, and the residue was recrystallized for precipitation with toluene. The precipitate was filtered and cleaned with toluene, obtaining 6.2 g of a compound (yield: 83.5%).

(Calculation value: 816.99/Measurement value: MS[M+1]816)

### Example 3

### Synthesis of Compound 283

The compound 283, as an example, was synthesized according to the following Reaction Scheme 3.

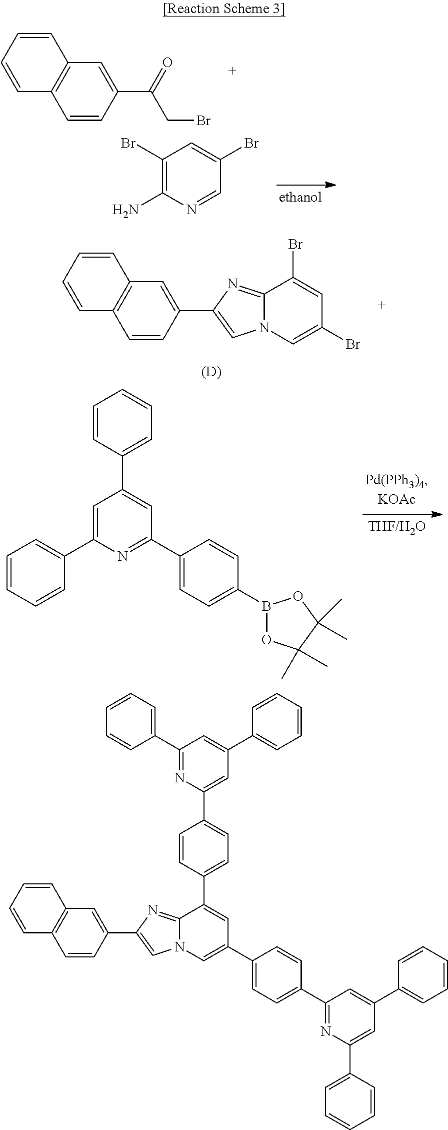

First Step; Synthesis of Intermediate Product (D)

24.9 g (100 mmol) of 2-bromo acetyl naphthalene and 25.2 g (100 ml) of 2-amino-3,5-dibromopyridine were suspended in 300 mL of ethanol. The suspension solution was heated and refluxed for 12 hours. After cooling down the reactant, the precipitated solid was filtered and cleaned with ethanol, obtaining 26.8 g of an intermediate product (D) (yield: 66%).

Second Step: Synthesis of Compound 283

3.5 g (8.7 mmol) of the intermediate product (D), 9.4 g (21.7 mmol) of the compound (C), and 0.5 g (0.44 mmol) of tetrakis-(triphenylphosphine)palladium were suspended in 400 mL of tetrahydrofuran and a solution prepared by dissolving 4.8 g (34.8 mmol) of potassium acetate in 200 mL of water. The mixture was heated and refluxed for 12 hours. The liquid reactant was separated into two layers, and an organic layer thereof was cleaned with a sodium chloride saturated aqueous solution and dried with anhydrous sodium sulfate. Then, an organic solvent was distilled and removed under a reduced pressure, and its residue was recrystallized with tetrahydrofuran/methanol. The precipitate was filtered and separated and then, cleaned with methanol, obtaining 6.1 g of a compound (yield: 82%).

(Calculation value: 855.03/Measurement value: MS[M+1]855)

### Example 4

### Synthesis of Compound 318

The compound 318, as an example, was synthesized according to the following Reaction Scheme 4.

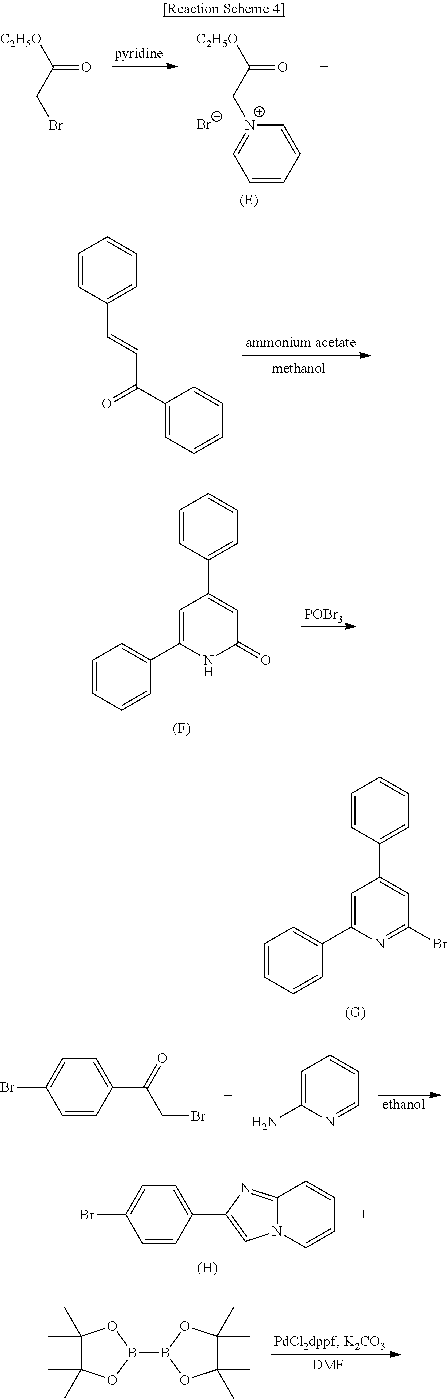

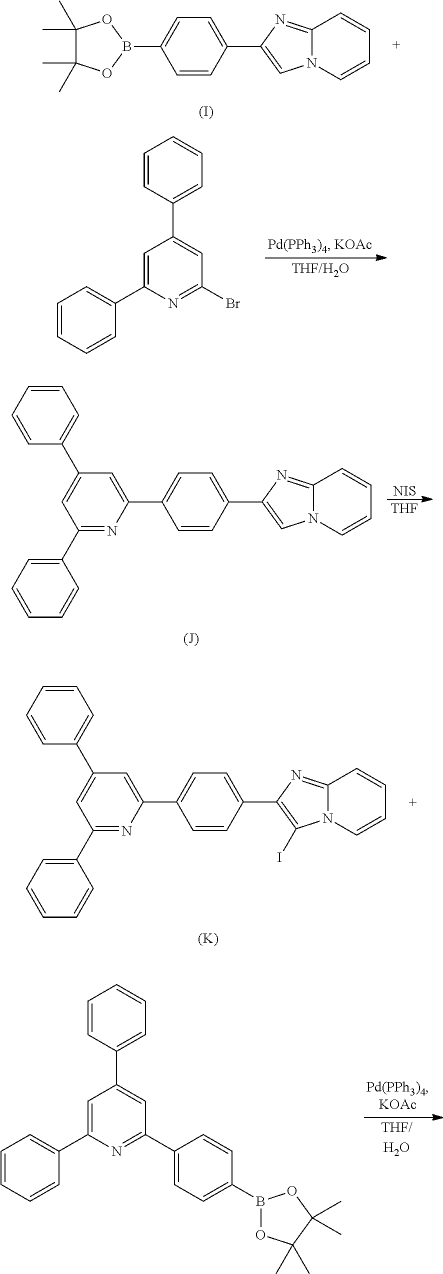

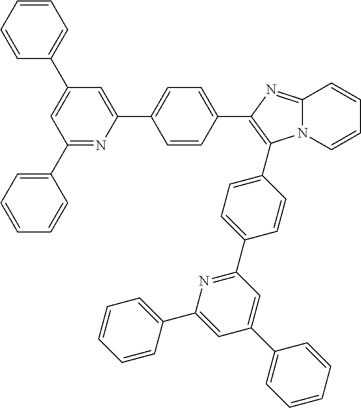

First Step; Synthesis of Intermediate Product (E)

50 mL (451 mmol) of ethylbromoacetate was slowly put into 700 mL of pyridine. The mixture was agitated at room temperature for 2 hours. The precipitated solid therein was filtered and separated and cleaned with diethyl ether, obtaining 105 g of an intermediate product (E) (yield: 94%).

Second Step; Synthesis of Intermediate Product (F)

42.5 g (172.9 mmol) of the intermediate product (E), 30 g (144 mmol) of transchalcone, and 111 g (1440 mmol) of ammonium acetate were suspended in 600 mL of methanol, and the mixture was heated and refluxed for 12 hours. After cooling down the reactant, the precipitated solid therein was filtered and separated and cleaned with methanol, obtaining 831.2 g of an intermediate product (F) (yield: 87%).

Third Step; Synthesis of Intermediate Product (G)

20 g (80.9 mmol) of the intermediate product (F) and 25 g (87.2 mmol) of phosphoryl tribromide were agitated together at 130° C. for 2 hours. The liquid reactant was cooled down to room temperature, and water was poured for neutralization thereto. A solid produced therein was filtered. The obtained solid was cleaned with methanol, obtaining 17.3 g of an intermediate product (G) (yield: 68%).

Fourth Step; Synthesis of Intermediate Product (H)

50 g (180 mmol) of 4-bromophenacyl bromide and 20.3 g (220 ml) of 2-aminopyridine were suspended in 300 mL of ethanol. The suspension reactant was heated and refluxed for 12 hours. After cooling down the reactant, the precipitated solid produced therein was filtered and separated and cleaned with ethanol, obtaining 36.6 g of an intermediate product (H) (yield: 74%).

Fifth Step; Synthesis of Intermediate Product (I)

27.3 g (100 mmol) of the intermediate product (H), 30.5 g (120 mmol) of bis(pinacolato)diboron, 0.82 g (1 mmol) of [1,1′-bis(diphenylphosphino)ferrocene]dichloropalladium (II), and 29.4 g (300 mmol) of potassium carbonate were suspended in 250 mL of dimethylformamide and agitated together at 80° C. for 12 hours. After cooling down the reaction solution, it was poured down to distilled water for precipitation. The precipitated solid was filtered and separated. The filtered solid was recrystallized with ethylacetate/hexane, obtaining 22.6 g of an intermediate product (I) (yield: 70%).

Sixth Step; Synthesis of Intermediate Product (J)

9.1 g (25.8 mmol) of the intermediate product (I), 8 g (21.7 mmol) of the compound (G), and 0.9 g (0.8 mmol) of tetrakis-(triphenylphosphine)palladium were suspended in 200 mL of tetrahydrofuran, and a solution prepared by dissolving 7.1 g (51.6 mmol) of potassium acetate in 100 mL of water. The mixture was heated and refluxed for 12 hours. The liquid reactant was separated into two layers. The organic layer therein was cleaned with a sodium chloride saturated aqueous solution and dried with anhydrous sodium sulfate. Then, an organic solvent therein was distilled and removed under a reduced pressure, and its residue was separated with a silica gel column, obtaining 3.35 g of a compound (J) (yield: 30%).

Seventh Step; Synthesis of Intermediate Product (K)

3.3 g (7.8 mmol) of the intermediate product (J) and 2.1 g (9.4 mmol) of N-iodosuccinimide were dissolved in 150 mL of tetrahydrofuran, and the solution was agitated at 50° C. for 12 hours. After cooling down the reactant, its solvent was distilled under a reduced pressure. The obtained solid was dissolved in dichloromethane, and the solution was reprecipitated in methanol and filtered, obtaining 4.3 g of a compound (K) (yield: 100%).

Eighth Step: Synthesis of Compound 318

4.3 g (7.8 mmol) of the intermediate product (K), 4.1 g (9.4 mmol) of the compound (C), and 0.27 g (0.23 mmol) of tetrakis-(triphenylphosphine)palladium were suspended in 400 mL of tetrahydrofuran, and a solution prepared by dissolving 2.2 g (17.2 mmol) of potassium acetate in 200 mL of water was added thereto. The mixture was heated and refluxed for 12 hours. The liquid reactant was separated into two layers, and the organic layer therein was cleaned with a sodium chloride saturated aqueous solution and dried with anhydrous sodium sulfate. Then, an organic solvent therein was distilled and removed under a reduced pressure, and its residue was separated with a silica gel column, obtaining 5.36 g of a compound (yield: 93%).

(Calculation value: 728.88/Measurement value: MS[M+1]728)

### Example 5

### Synthesis of Compound 177

The compound 177, as an example, was synthesized according to the following Reaction Scheme 5.

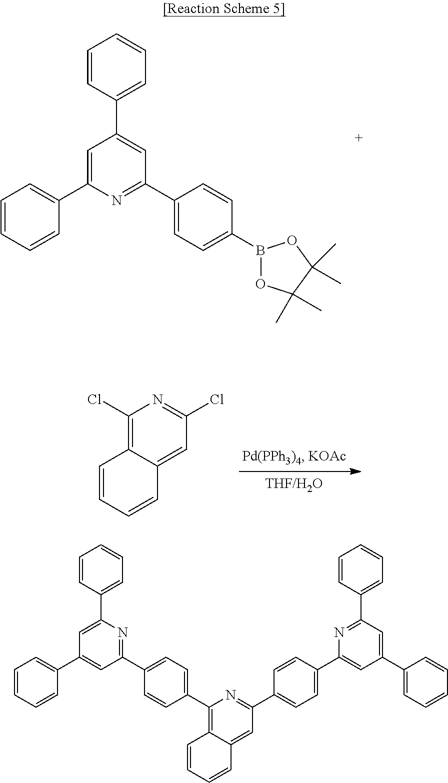

3 g (15.1 mmol) of 1,3-dichloroisoquinoline, 16.4 g (37.8 mmol) of the compound (C), and 0.9 g (0.76 mmol) of tetrakis-(triphenylphosphine)palladium were suspended in 180 mL of tetrahydrofuran, and a solution prepared by dissolving 8.3 g (60.4 mmol) of potassium acetate in 90 mL of water was added thereto. The mixture was heated and refluxed for 12 hours. The liquid reactant was separated into two layers, and an organic layer therein was cleaned with a sodium chloride-saturated aqueous solution and dried with anhydrous sodium sulfate. Then, an organic solvent therein was distilled under a reduced pressure, and its residue was recrystallized with chlorobenzene, obtaining 5.4 g of a compound (yield: 48%).

(Calculation value: 739.90/Measurement value: MS[M+1]739)

A glass transition temperature and a thermal decomposition temperature of the synthesized materials were measured using DSC and TGA.

(Fabrication of Organic Photoelectric Device)

### Example 6

An organic photoelectric device was fabricated using 1000 Å-thick ITO as an anode and 1000 Å-thick aluminum (Al) as a cathode.

In particular, the anode was prepared by cutting an ITO glass substrate having a sheet resistance of 15 Ω/cm² into a size of 50 mm×50 mm×0.7 mm and cleaning the cut ITO glass substrate in acetone, isopropyl alcohol, and pure water, respectively for 5 minutes and with UV ozone for 30 minutes.

Next, N1,N1′-(biphenyl-4,4′-diyl)bis(N-1-(naphthalen-2-yl)-N4,N4-diphenylbenzene-1,4-diamine) was deposited to be 65 nm thick as a hole injection layer (HIL) on the glass substrate, and N,N-di(1-naphthyl)-N,N′-diphenylbenzidine was deposited to be 40 nm thick as a hole transport layer (HTL).

Then, 5% of N,N,N′,N′-tetrakis(3,4-dimethylphenyl)chrysene-6,12-diamine and 95% of 9-(3-(naphthalen-1-yl)phenyl)-10-(naphthalen-2-yl)anthracene were deposited to be 25 nm thick as an emission layer on the hole transport layer (HTL).

Then, the compound according to Example 1 was deposited to be 30 nm thick on the emission layer as an electron transport layer (ETL).

On the electron transport layer (ETL), Liq was vacuum-deposited to be 0.5 nm thick on the electron injection layer (EIL), and Al was vacuum-deposited to be 100 nm thick, forming a Liq/Al electrode.

### Example 7

An organic photoelectric device was fabricated according to the same method as Example 6, except for using the compound according to Example 2 instead of the compound according to Example 1 to form an electron transport layer (ETL).

### Example 8

An organic photoelectric device was fabricated according to the same method as Example 6, except for using the compound according to Example 4 instead of the compound according to Example 1 to form an electron transport layer (ETL).

### Comparative Example 1

An organic photoelectric device was fabricated according to the same method as Example 6, except for using 35 nm-thick tris(8-hydroxy quinolato)aluminum (Alq3) instead of the compound according to Example 1 to form an electron transport layer (ETL).

Performance measurement of Organic Photoelectric Device

### Experimental Examples

### Measurement Method

Each of the obtained organic photoelectric devices according to Examples 6 to 8 and Comparative Example 1 was measured for luminance change, current density change depending upon the voltage, and luminous efficiency. The specific method was as follows.

1) Measurement of Current Density Change Depending on Voltage Change

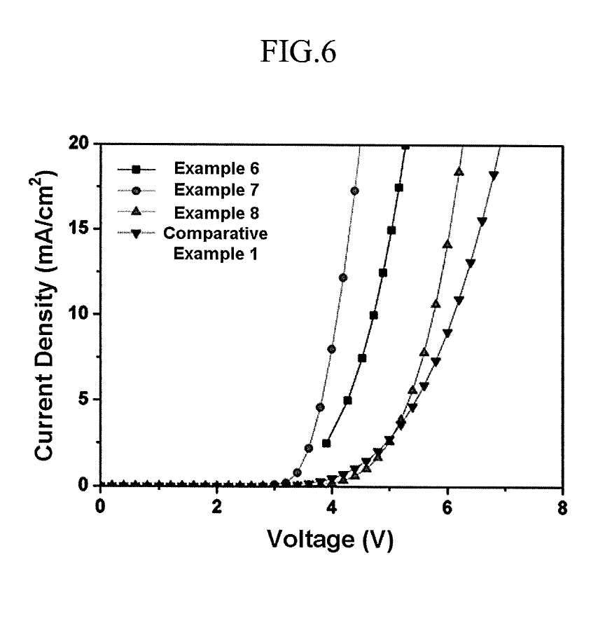

The obtained organic photoelectric devices were measured for current values flowing in the unit devices while increasing the voltages using a current-voltage meter (Keithley 2400), and the measured current values were divided by an area to provide the results. The results are shown in FIG. 6.

2) Measurement of Luminance Change Depending on Voltage Change

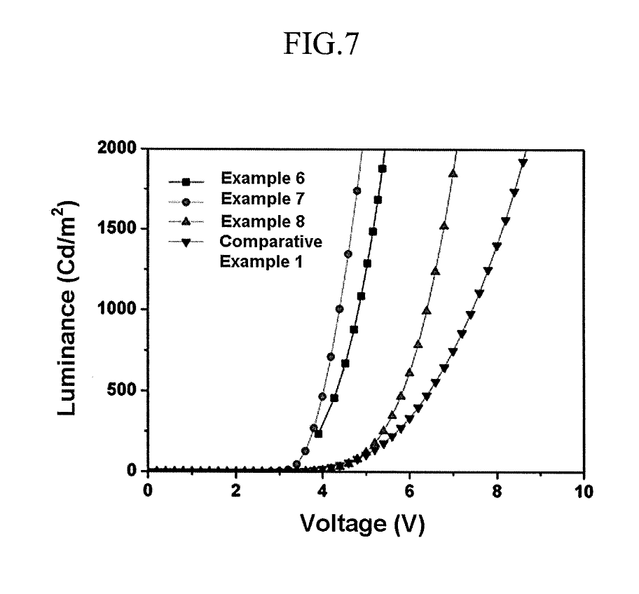

The obtained organic photoelectric devices were measured for luminance using a luminance meter (Minolta Cs-1000A) while increasing the voltage. The results are shown in FIG. 7.

3) Measurement of Luminous Efficiency and Electric Power Efficiency

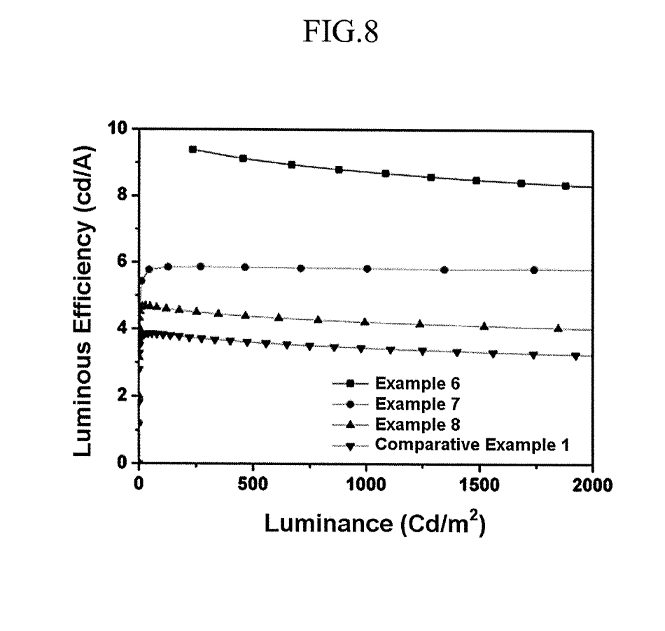

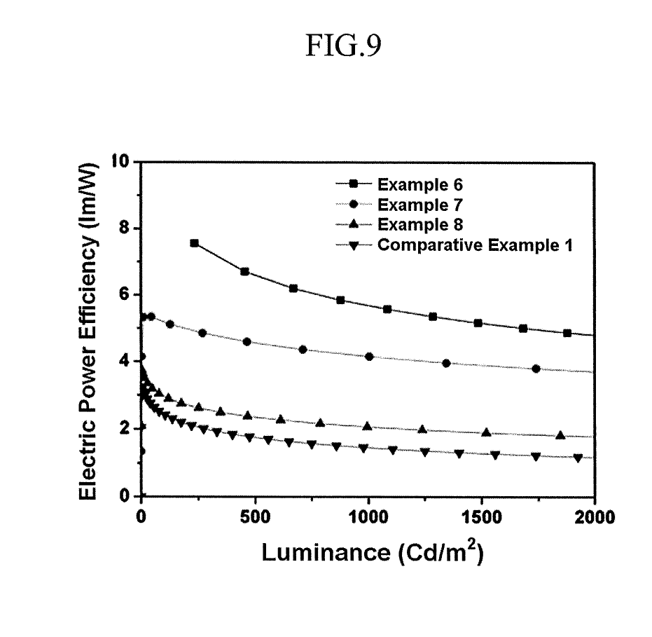

Current efficiency and electric power efficiency were calculated by using luminance and current density from 1) and 2) and voltage. The results are shown in FIGS. 8 and 9, and Table 12.

4) Measurement of Life-Span

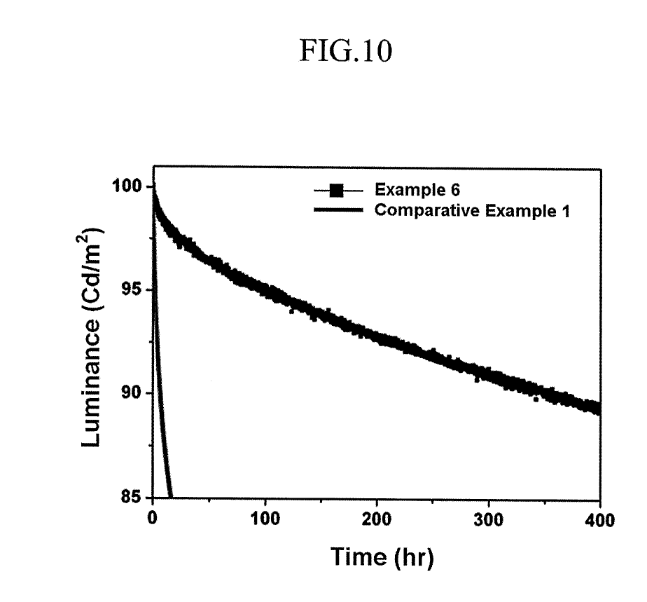

The organic photoelectric devices according to Example 6 and Comparative Example 1 were compared about life-span by decreasing luminance depending on time, referring to a reference luminance of 1000 cd/m². The results are shown in FIG. 10.

Results

As shown in FIG. 6, the organic photoelectric devices according to Examples 6 to 7 had remarkably better current density than the organic photoelectric device according to Comparative Example 1 at the same voltage. A larger current density difference was shown at a higher high voltage.

In addition, as shown in FIG. 7, the organic photoelectric devices according to Examples 6 to 7 had remarkably better light emitting luminance than the organic photoelectric device according to Comparative Example 1 at the same voltage. A larger light-emitting luminance difference was shown at a higher voltage.

As shown in FIGS. 8 and 9 and the following Table 12, the organic photoelectric devices according to Examples 6 and 7 had remarkably excellent luminous efficiency and electric power efficiency compared with the organic photoelectric device according to Comparative Example 1.

TABLE 12

| | Luminance at 500 cd/m² | | |
|---|---|---|---|
| | | Luminous | Electric power |
| | Driving | efficiency | efficiency |
| | voltage (V) | (cd/A) | (lm/W) |
| Example 6 | 4.3 | 9.1 | 6.7 |
| Example 7 | 4 | 5.85 | 4.59 |
| Example 8 | 5.8 | 4.39 | 2.38 |
| Comparative | 6.6 | 3.58 | 1.70 |
| Example 1 | | | |

As shown in FIG. 10, the organic photoelectric device according to Example 6 had greater than or equal to 20 times longer life-span than the organic photoelectric device according to Comparative Example 1.

Organic photoelectric devices including the compounds according to embodiments disclosed herein showed a low driving voltage and a high luminous efficiency, and thus, an increased life span of a device, which was confirmed by device operation experiments.

By way of summation and review, examples of the organic photoelectric devices may include an organic light emitting diode, an organic solar cell, an organic photo conductor drum, an organic transistor, and the like. Such devices may utilize a hole injecting or transporting material, an electron injecting or transporting material, or a light emitting material. Particularly, organic light emitting diodes (OLEDs) have recently drawn attention due to an increasing demand for flat panel displays.

In general, the term “organic light emission” may be used to refer to a transformation of electrical energy to photo-energy. An organic photoelectric device such as an OLED transforms electrical energy into light by applying a current to an organic light emitting material. An OLED may have a structure such that a functional organic material layer is interposed between an anode and a cathode. The organic material layer may have a multi-layer structure respectively including different materials, for example, a hole injection layer (HIL), a hole transport layer (HTL), an emission layer, an electron transport layer (ETL), and an electron injection layer (EIL) in order to improve efficiency and stability of an organic photoelectric device.

In such an organic photoelectric device, when a voltage is applied between an anode and a cathode, holes from the anode and electrons from the cathode are injected to an organic material layer and recombined to generate excitons having high energy. The generated excitons generate a light having a certain wavelength while shifting to a ground state. Light emitting materials of an organic material layer may be classified as blue, green, and red light emitting materials according to emitted colors and yellow and orange light emitting materials to emit colors approaching natural colors. Various materials have been used as light emitting materials including a low molecular aromatic diamine and aluminum complex as an emission layer forming material. Specifically, such an emission layer has a structure that a diamine derivative thin film (hole transport layer (HTL)) and a tris(8-hydroxy-quinolate)aluminum (Alq3) thin film are laminated. Moreover, a phosphorescent light emitting material has been used for a light emitting material of an organic light emitting diode. A phosphorescent material emits lights by transiting electrons from a ground state to an exited state, non-radiance transiting a singlet exciton to a triplet exciton through intersystem crossing, and transiting a triplet exciton to a ground state to emit a light.

A maximum light emitting wavelength of a light emitting material may be shifted to a longer wavelength and color purity of emitted light may be decreased because of interactions among molecules. Moreover, device efficiency may deteriorate because of light emitting quenching effects. Accordingly, a host/dopant system may be used in a light-emitting layer in order to improve color purity and increase luminous efficiency and stability through energy transfer.

In order to implement the aforementioned excellent performances of an organic photoelectric device, it is desirable to provide a stable and efficient material, a material constituting an organic material layer, for example, a hole injection material, a hole transport material, a light emitting material, an electron transport material, an electron injection material, and a light emitting material such as a host and/or a dopant, should be first supported by a stable and efficient material.

Embodiments disclosed herein may provide a compound for an organic photoelectric device having an excelling life span, efficiency, electrochemical stability, and thermal stability. An organic photoelectric device including a compound according to an embodiment may have excellent life-span characteristics and a high luminous efficiency at a low driving voltage may be provided due to the excellent electrochemical and thermal stability of the compound used in the organic photoelectric device.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope as set forth in the following claims.

What is claimed is:

1. A compound for an organic photoelectric device, the compound being represented by the following Chemical Formula 1: 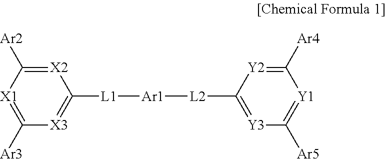
wherein, in Chemical Formula 1,
Ar1 is a substituted or unsubstituted C2 to C30 heteroarylene group,
Ar1 to Ar5 are independently a substituted or unsubstituted C6 to C30 aryl group,
L1 and L2 are independently a substituted or unsubstituted C6 to C30 arylene group,
X1 to X3 are independently a heteroatom or C—H, provided that at least one of X1 to X3 is a heteroatom,
Y1 to Y3 are independently a heteroatom or C—H, provided that at least one of Y1 to Y3 is a heteroatom, and
wherein Ar1 is selected from the group of a substituted or unsubstituted imidazolylene group, a substituted or unsubstituted thiazolylene group, a substituted or unsubstituted oxazolylene group, a substituted or unsubstituted oxadiazolylene group, a substituted or unsubstituted triazolylene group, a substituted or unsubstituted pyrimidinylene group, a substituted or unsubstituted pyridinylene group, a substituted or unsubstituted pyradazinylene group, a substituted or unsubstituted quinolinylene group, a substituted or unsubstituted isoquinolinylene group, a substituted or unsubstituted acridinylene group, a substituted or unsubstituted imidazopyridinylene group, and a substituted or unsubstituted imidazopyrimidinylene group.

2. The compound as claimed in claim 1, wherein Ar1 is a substituent selected from the group of the following Chemical Formulae 2 to 7: 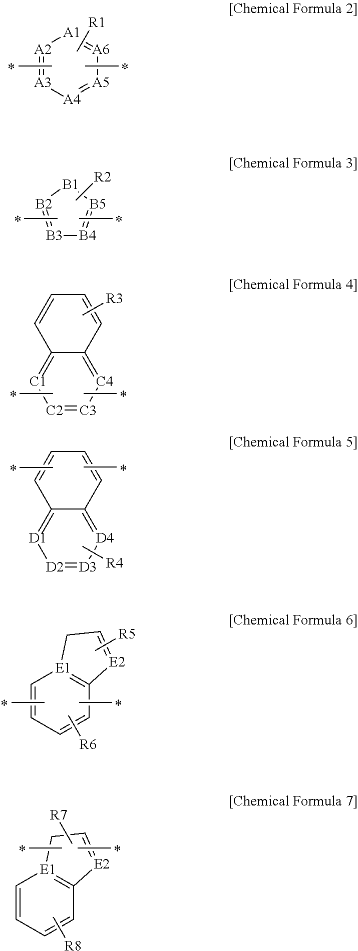
wherein, in Chemical Formulae 2 to 7,
A 1 to A6 are independently a heteroatom or C—H, provided that at least one of A1 to A6 is a heteroatom,
B1 to B5 are independently a heteroatom or C—H, provided that at least one of B1 to B5 is a heteroatom,
C1 to C4 are independently a heteroatom or C—H, provided that at least one of C1 to C4 is a heteroatom,
D1 to D4 are independently a heteroatom or C—H, provided that at least one of D1 to D4 is a heteroatom,
E1 and E2 are independently a heteroatom or C—H, provided that at least one of E1 and E2 is a heteroatom, and
R1 to R8 are independently selected from the group of hydrogen, a substituted or unsubstituted C1 to C10 alkyl group, a substituted or unsubstituted C5 to C30 aryl group, and a substituted or unsubstituted C2 to C30 heteroaryl group.

3. The compound as claimed in claim 1, wherein Ar2 to Ar5 are independently selected from the group of a substituted or unsubstituted phenyl group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted perylenyl group, and a substituted or unsubstituted chrysenyl group.

4. The compound as claimed in claim 1, wherein L1 and L2 are independently selected from the group of a substituted or unsubstituted phenylene group, a substituted or unsubstituted naphthylene group, a substituted or unsubstituted anthracenylene group, a substituted or unsubstituted phenanthrenylene group, a substituted or unsubstituted pyrenylene group, a substituted or unsubstituted perylenylene group, and a substituted or unsubstituted chrysenylene group.

5. An organic photoelectric device, comprising
an anode, a cathode, and an organic thin layer between the anode and the cathode,
wherein the organic thin layer includes the compound for an organic photoelectric device as claimed in claim 1.

6. The organic photoelectric device as claimed in claim 5, wherein the organic thin layer is selected from the group of an emission layer, a hole transport layer (HTL), a hole injection layer (HIL), an electron transport layer (ETL), an electron injection layer (EIL), a hole blocking layer, and a combination thereof.

7. The organic photoelectric device as claimed in claim 5, wherein the compound for an organic photoelectric device is included in an electron transport layer (ETL) or an electron injection layer (EIL).

8. The organic photoelectric device as claimed in claim 5, wherein the compound for an organic photoelectric device is included in an emission layer.

9. The organic photoelectric device as claimed in claim 5, wherein the compound for an organic photoelectric device is used as a phosphorescent or fluorescent host material in an emission layer.

10. The organic photoelectric device as claimed in claim 5, wherein the compound for an organic photoelectric device is used as a fluorescent blue dopant material in an emission layer.

11. The organic photoelectric device as claimed in claim 5, wherein the organic photoelectric device is selected from the group of an organic light emitting diode, an organic solar cell, an organic transistor, an organic photo conductor drum, and an organic memory device.

12. A display device including the organic photoelectric device as claimed in claim 5.
